(19) United States
(12) Patent Application Publication
(10) Pub. No.: US 2015/0196694 A1
(43) Pub. Date: Jul. 16, 2015

(54) Directional Bandage for Delivering Oxygenated Solution to Wound and Method of Treating a Wound Using Same

(71) Applicants: C. Edward Eckert, New Kensington, PA (US); Lewis V. Whitman, Mars, PA (US); Scott Gene Fisher, Fombell, PA (US)

(72) Inventors: C. Edward Eckert, New Kensington, PA (US); Lewis V. Whitman, Mars, PA (US); Scott Gene Fisher, Fombell, PA (US)

(21) Appl. No.: 14/558,228

(22) Filed: Dec. 2, 2014

Related U.S. Application Data
(60) Provisional application No. 61/910,573, filed on Dec. 2, 2013.

(51) Int. Cl. A61M 1/00; A61F 13/02; A61F 13/00

(52) U.S. Cl. CPC A61M 1/0088; A61M 1/0084; A61M 1/0058; A61F 13/00063; A61F 13/0266; A61M 2202/0476; A61M 2202/048; A61M 2202/30; A61M 2202/0208; A61M 2202/0225; A61M 2202/0275; A61M 2202/0283; A61M 2205/3368; A61M 2205/3673

(57) ABSTRACT

Dispersing a gas into solution for intentionally directional inflow and outflow through a bandage to a human tissue wound for treatment of said wound with the gas-containing solution. Methods of using the solution to treat a wound are provided. The gas molecules may be mostly oxygen and/or contain supplemental additions of carbon dioxide, nitrous or nitric oxide.

### CROSS-REFERENCE TO RELATED APPLICATION

This application is a perfection of Provisional Patent Application Ser. No. 61/910,573, filed Dec. 2, 2013, the disclosure of which is fully incorporated by reference herein.

### BACKGROUND OF INVENTION

There is growing interest in the medical field for providing irrigation into and drainage from human wounds to the skin and adjacent regions. Bandages for the treatment of chronic skin ulcers are but one example of applications for these wound coverings. A whole industry has emerged around this philosophy evidenced by the popularity of KCI's well established, and highly patent protected, system of wound V.A.C., negative pressure wound therapies. See, generally, www.kci1.com

Various manufacturers have sought to permit moist wound healing, either through lavage by a connecting tube extending through dressing into a wound site, or by sealing the wound site with a dressing that is liquid impermeable. To efficiently accomplish various methods of ulcer and wound therapy with a plastic film bandage, an effective plastic film bandage is adhered position onto the patient in aseptic condition. While plastic film bandages are gas permeable, they tend to be thin and not self-supporting. Thus, they wrinkle quite easily and should be supported by some means to remain unwrinkled until applied to the desired site.

In one plastic film bandage design, a peripheral frame supports a peel-away center portion that is at least partially transparent for wound viewing to better position the bandage there over.

This invention provides an improved device (bandage) for delivering an improved liquid solution directly to one or more critical areas of a wound, for treatment and more rapid healing of that wound proper. Further, this invention uses such devices/bandages for better imparting to a given body treatment region/area a solution with quantities of dissolved gas via a dispersion of small micro-bubbles suspended in that solution. Such micro-bubbles may have a diameter of less than about 125 microns, and preferably less than 50 microns.

In the context of this invention, it is desirable to minimize the terminal velocity of such micro-bubbles in the suspending liquid, typically water or other suitable medicinal flushing composition. Since the residence time of a bubble in a liquid is inversely proportional to the square of the bubble diameter, such micro-bubbles will have an extended contact time with a fluid as compared to a system consisting of larger diameter bubbles. This quality is known as gas holdup. Additionally, the micro-bubble/liquid interfacial area is inversely proportional to the square of the bubble diameter. Therefore, it will be shown that a solution of a solute gas such as oxygen in a liquid solvent like water can be manipulated to produce a dispersion of micro-bubbles consisting of solute gas nucleated and precipitated from the liquid. This results in extended residence time and a high interfacial area that maximizes the contact between the gas (as micro-bubbles) and the surface (i.e. human tissue) with which it comes in intimate contact.

Various means of exposing living tissue to oxygen are known. Most notably, hyperbaric chambers employ a pure oxygen atmosphere typically pressurized to under 3 atmospheres. In addition, certain oxygen tents are intended to operate at or near atmospheric pressure. Use of the latter oxygen tents has known benefits. Cutaneous oxygen uptake through the skin surface can be locally and systemically beneficial by augmenting respiration as an oxygen uptake mechanism. Since cutaneous uptake does not depend on blood circulation through capillaries, local oxygen uptake through the extremities can be beneficial in providing tissue health. A generally enhanced disposition and accelerated recovery from physical exertion can result. Inside of a bandaged wound, this invention will create a similar or enhanced condition for accomplishing these and still other benefits without the accumulation of oxygen gas in a “tent environment”.

The effect of oxygen on living tissue can be characterized by three regimes: metabolic enhancement (growth accelerator), metabolic inhibition (growth arrest), and toxicity. In the former, oxygenated solutions with micro-bubble suspensions should accelerate the healing and regeneration rate of damaged tissue. Such wounds include cuts, lacerations, sores and burns on the face, arms, legs and torso. When wounds begin to heal, fibroblastic cells divide and spread throughout the wound area. Such cells produce collagen, an important protein that facilitates healing. Supplying sufficient quantities of oxygen to the wound area will significantly enhance fibroblast proliferation. In particular, the fibroblastic cells use amino acids hydroxylated with oxygen to synthesize collagen chains.

The amount of oxygen initially dissolved into solution for dispersal through the preferred directional bandage described herein can vary somewhat with the method used to dissolve gas into solution. Such methods generally comprise two steps: creating a solute gas/solvent liquid interfacial area; then exposing the gas/liquid mixture to an elevated pressure. The former step affects the kinetics or rate at which the solution process occurs while the latter determines the maximum theoretical dissolved. Small bubbles create interfacial area and promote more favorable kinetics. The second step is a pressure-concentration relationship, such as Henry's Law for dilute solutions and Sievert's Law for diatomic gases at higher concentrations. These steps may be combined, although the source of oxygen must operate at a higher final pressure rather than allowing a pump, for example, to pressurize both the liquid and gas components after the gas has been introduced.

One method for oxygenating water is the coarse bubble aeration process, a subset of methods categorically known as “air diffusion”. By that method, pressurized air or oxygen gas is introduced through a pipe with small holes submerged in a container of water. At a gas pressure sufficient to overcome hydrostatic head pressure, pressure losses during passage through these small orifices will be sustained. As a result, bubble aeration occurs at relatively low pressures that are predominantly a function of tube immersion depth and density of the liquid.

Since all interphase interfaces have a characteristic surface energy, the creation of interfacial (surface) area is an energetic process. As a gas passes through an orifice, for example, pressure energy is converted to kinetic energy, which consequently satisfies the energetic requirements of the system for the production of surface area. Area and velocity are inversely proportional; hence, as the orifice diameter decreases, the corresponding pressure drop and gas velocity increase, and more surface area is generated. Smaller bubbles result. This process has a limiting condition, however. The amount of heat (as irreversible work) that is produced is inversely proportional to the square of orifice diameter. It therefore becomes impractical and energetically inefficient to operate at exceptionally small orifice diameters. This process also has an absolute limit as a gas velocity of Mach 1 is approached within the pore of a porous medium used to create bubbles. Because a pore lacks the convergent/divergent geometry required to achieve supersonic flow, increasing pressure beyond the critical pressure will not result in a further reduction of bubble size.

Since oxygen is introduced into solution at relatively low pressures in the coarse bubble aeration process, the bubbles themselves tend to be relatively large and the aggregate bubble surface area for their dispersion relatively small. The limited surface area produced by bubble aeration limits the rate at which gas can be dissolved and practically limits the concentration of gas that can be dissolved into solution. Oxygen dissolution is a function of the interfacial contact area between gas bubbles and the surrounding medium, and bulk fluid transport (mixing) in the liquid phase. In particular, the rate of oxygen dissolution is directly proportional to the surface area of the bubbles. A dispersion of very small bubbles, i.e., those having diameters of about 50 microns, will have a much larger total surface area than a dispersion of large bubbles occupying the same volume. Consequently, the rate of oxygen dissolution in bubbling aeration is limited by the size of the bubbles introduced into the solvent.

Oxygen dissolution in bubbling aeration is also limited by ambient pressure conditions above the solution. If the solution being aerated is exposed to atmospheric conditions, the dissolved oxygen concentration will be limited to the solubility limit of oxygen (at its partial pressure in air of 0.21 atm.) under such conditions. The desirability of bubbling aeration can be further hampered by equipment and energy requirements. Large blower units can be used to force gas into a carrying liquid. Such blowers generate high-energy costs and often require special soundproof installations or other engineering costs. Bubble aeration is therefore an impractical process for producing oxygenated solutions or solution/suspensions for health related applications.

Other methodologies have been used to prepare oxygenated solutions based on pressure tanks and adaptations of carbonator devices that dissolve carbon dioxide in water. For a given pressure and temperature, the solubility of carbon dioxide in water exceeds that of oxygen by over an order of magnitude. Carbonators therefore are acceptable for preparing carbonated water solutions but not oxygenated solutions.

In this invention, conditions most favorable to produce a dispersion of small diameter micro-bubbles in a suspending solvent liquid with high interfacial area are created either at elevated pressure or with a subsequent pressure increase. The elevated pressure environment will dissolve gas into the liquid, since the concentration of gas in solution and pressure over that solution are directly related.

### SUMMARY OF THE INVENTION

It is an object of this invention to provide a novel system for delivering dissolving gas in a liquid directly to a human wound area for more intimate treatment of that wound area. It is another object to dissolve large quantities of molecular oxygen in water for providing a solution saturated with metastable oxygen, creating a holdup of suspended micro-bubbles in that solution before, preferentially, directionally delivering that solution proximate the very wound site to be treated.

In accordance with these objects, there is provided a method for delivering dispersed gas in a liquid directly to the site of a bandaged wound, by covering that wound with a device having means for precisely focusing one or more streams of such solution to areas where greatest benefit should be realized. One method of gas dispersion comprises: providing a liquid pumping means and liquid for introducing to the liquid pumping means, then pumping that liquid to greater than 0.8 atmospheres. Next, a gas (like oxygen) is introduced to that pressurized liquid causing the gas to disperse therein in bubble form. The solution is then subjected to “shearing” for reducing bubble size to as low as 5 μm diameter in the hypersaturated liquid.

Based on the foregoing, an oxygenated mixture is provided having dissolved molecular oxygen content well above the equilibrium limit at ambient conditions. That mixture can be delivered to skin tissue in a non-traumatic medium. Since dissolution of oxygen occurs under hyperbaric conditions, a large concentration of oxygen may be dissolved therein. The resulting solution can have dissolved O₂ contents as high as 2000 mg/l. As that solution circulates through the preferred covering (bandage), the dissolved oxygen nucleated into fine bubbles will better attach to skin fragments and adjacent wound areas. Additional energy, as produced by ultrasonic means, can be imparted to facilitate further nucleation of gas bubbles.

It should be noted that O₂ is a molecule (O:O) with shared electrons and that O⁻ is an ion (free radical) with an unpaired electron. Water is formed from free radicals: H⁺+(OH)⁻═H₂O. Free radicals are usually reactive oxygen free radicals which can form from, for example, hydrogen peroxide: H₂O₂→H₂O+O⁻ or ozone: O₃═O₂+O⁻.

This method can also be practiced with any solute/dispersion gas and liquid solvent that is provides benefit to living tissue and/or the host. It may be beneficial, for example to use carbon dioxide as the gas phase . . . possibly to help exfoliate necrotic skin. Carbon dioxide is desirable in this case because of a substantially higher solubility in water as compared to oxygen. During the nucleation of the gas phase to form micro-bubbles, a significantly greater volume of carbon dioxide gas would be available to form micro-bubbles as compared to oxygen since dissolved gas is the source of the dispersed gas.micro-bubbles.

In this instance, the clear solution containing carbon dioxide and water will be first allowed to infiltrate the necrotic skin. Once infiltrated, the solution would be energized using thermal or mechanical energy to cause gas to nucleate and precipitate from solution. In the case of carbon dioxide, a volume change greater than 25:1 will occur. Such an expansion is an energy source for exfoliating necrotic skin that has been infiltrated by the original solution.

As a further option, solutions to which nitrous and/or nitric oxides are purposefully added may be used as a substitute or supplement to the aforesaid oxygen-based bandage immersion solutions. Furthermore, one or more compatible antibiotics (or possibly even some localized liquid anesthetics/pain killers may be blended therein before added to the solution that directionally flushes into a human wound treatment area.

### BRIEF DESCRIPTION OF DRAWINGS

FIG. 1A is a top plan view of one embodiment of bandage over a wound according to this invention;

FIG. 1B is a top plan view of an alternative embodiment showing a bandage with at least two solution inlets;

FIG. 2A is a side sectional view of a tube configuration with a flexible plastic directional tip;

FIG. 2B is a side sectional view of a first alternative tip configuration with an internal stiffener element; and

FIG. 2C is a side sectional view of a second alternative tip configuration with an external stiffener element.

FIG. 3A is a top view of a third alternative tip configuration with two separate spray exits; and

FIG. 3B is a top close up view of a fourth alternative tip configuration with multiple dispensing apertures.

### DESCRIPTION OF PREFERRED EMBODIMENTS

In the accompanying drawings, there are shown several embodiments of the invention. Therein, common elements are commonly numbered in the respective views. For the alternative embodiments, common elements are consistently numbered though in the next hundred series.

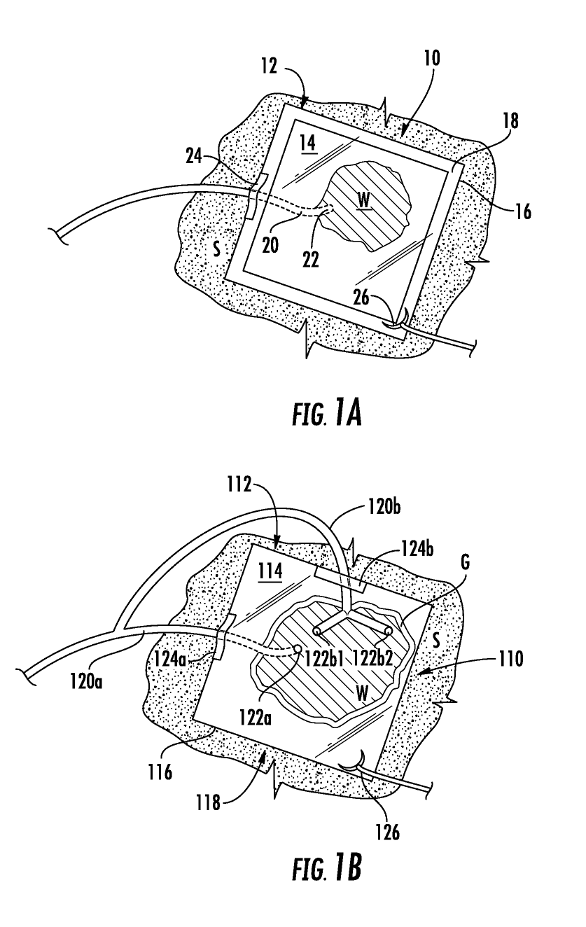

Referring now to FIG. 1A, there is shown a directional bandage, generally 10, according to a first preferred embodiment. That bandage 10 has an interior 12 and an exterior 14 with a full perimeter 16 thereabout. On at least the entire perimeter of interior 12, there is provided a waterproof adhesive strip 18 for attaching to the skin S of a patient being treated. On a less preferred basis, the entirety of interior 12 made be coated with an adhesive layer provided the wound area to be treated with same is protected with a gel or other coating/cover so that the adhesive interior does not contact directly with same.

Preferably, bandage 10 is fully transparent or at least partially or semi-transparent. With greater visibility, the bandage proper can be accurately situated directly over a human wound region W to be treated.

Each directional bandage unit includes at least one fluid delivery portal 20 having an outlet 22 through which the gas-containing solution is sprayed onto, into or otherwise contacted with the wound W. To better secure delivery portal 20 beneath the exterior 14 of bandage 10, there may be rigid or semi-flexible clamps 24 for positioning to the bandage perimeter 16.

At another point beneath the same bandage 10, there is situated at least one drain sink 26, preferably at or near an opposite end (or corner) from outlet 22 of delivery portal 20. That drain sink 26 is used to remove solution from the covered wound area after it has been used for contacting same.

As for the solution (not shown) that passes through the aforementioned bandage, it contains a gas, such as molecular oxygen, dissolved in a solvent, such as water. It will comprise a suspension formed by a dispersion of micro-bubbles containing a gas, such as molecular oxygen. For purposes of this description, the solution will be described as containing pure molecular oxygen gas in water. However, it is intended that the mixture may contain other solute gases, additives (medical or non-medical) and/or solvents as discussed below.

The solution itself should be kept in a static condition possibly by storing “remotely” in a separate vessel (not shown). The micro-bubble dispersion itself primarily consists of molecular oxygen gas bubbles that have nucleated out of the solution.

One representative example of homogeneous solution will now be described in greater detail. The solubility limit of oxygen in water under equilibrium conditions with air (p_{O2}=0.21) at 77° F. is approximately 8.3 mg/l. When initially exposed to atmospheric conditions, this homogeneous solution will have a super-saturated or hyper-saturated molecular oxygen content, i.e., above the solubility limit of oxygen in water under atmospheric conditions. Preferably, that homogeneous solution will have a dissolved oxygen concentration above 20 mg/l at 1 atm., and 65° F. or higher. More preferably, the solution has a dissolved oxygen concentration above about 40 mg/l at 1 atm. and 65° F. or higher. As a result, the oxygen concentration in the solution 15 is not stable when exposed to atmospheric conditions.

The super-saturated or hyper-saturated molecular oxygen content in solution can be preserved by limiting agitation and preventing flow conditions that might otherwise facilitate ebullition of oxygen gases. A high dissolved oxygen content is maintained by storing the solution in a manner that limits or prevents desorption of the gas. For instance, the solution may be stored and distributed in sealed screw top containers constructed of glass or alternative materials impervious to oxygen diffusion at these high oxygen concentrations.

The solution preferably contains micro-bubbles having an average bubble diameter of about 2-9 μm, or 10-100 microns. Micro-bubbles in this size range provide a significantly larger surface area than a cluster of large bubbles containing the same volume of gas. The magnitude of this difference can be visualized by performing calculations for several bubble diameters at a constant volume of gas. The following calculations show the surface areas present for a single bubble, a plurality of one-inch diameter bubbles and a plurality of 50-micron or 5 μm diameter micro-bubbles, wherein each calculation is based on one cubic foot of gas. The value, r, is the radius of a single bubble, Vₒ is the volume of a single bubble, Aₒ is the surface area of a single bubble, and A is the aggregate surface area for the bubble formation:

a. Single Bubble:

Vₒ=4/3πr³; r=(3Vₒ/4π)^{1/3}

Thus, when Vₒ=1.00 ft³, r=0.62 ft. Therefore, the diameter of a single bubble containing 1.00 ft³ of gas=1.24 ft.

The surface area of this single bubble (A_{b}) is:

A_{b}=4πr²=4π(0.62 ft)²=4.83 ft²

b: For One Inch Bubbles:

r=0.50 inches=0.042 ft.
The volume of a single bubble (V_{b}) is:

V_{b}=4/3πr³=4/3π(0.042 ft)³=3.1×10⁻⁴ ft³/bubble

A_{b}=4πr²=4(0.042 ft)²=2.22×10⁻² ft²

The number of one inch bubbles (n_{b}) in a 1.00 ft³ volume of gas is:

n_{b}=Vₒ/V_{b}=1.00 ft³/3.1×10⁻⁴ ft³/bubble=3,224 bubbles

The surface area (Aₒ) of a 1.00 ft³ volume of gas comprised of one inch bubbles therefore is:

Aₒ=ΣA_{b}=n_{b}A_{b}=3,224(2.22×10⁻² ft)=71.43 ft²

c. For 50μ Micro-Bubbles:

r=25μ/3.05×10⁵μ/ft=8.2×10⁻⁵ ft

V_{b}=4/3πr³=4/3π(8.2×10⁻⁵ ft)³=2.31×10⁻¹² ft³

A_{b}=4πr²=4π(8.2×10⁻⁵ ft)²=8.45×10⁻⁸ ft²

n_{b}=Vₒ/_{Vb}=1.00 ft³/2.31×10¹² ft³=4.32×10¹¹ bubbles

Aₒ=n_{b}A_{b}=4.32×10¹¹(8.45×10⁻⁸ ft²)=36,504 ft²

d. For 5 μm Micro-Bubbles:

r=2.5μ/3.05×10⁵μ/ft=8.2×10⁻⁶ ft

V_{b}=4/3πr3=4/3(8.2×10⁻⁶ ft)³=2.31×10⁻¹⁵ ft³

A_{b}=4πr²=4(8.2×10⁻⁶ ft)²=8.45×10⁻¹⁰ ft²

n_{b}=Vₒ/V_{b}=1.00 ft³/2.31×10⁻¹⁵ ft³=4.32×10¹⁴ bubbles

Aₒ=n_{b}A_{b}=4.32×10¹⁴(8.45×10⁻¹⁰ ft ²) 365,800 ft²

Based on the foregoing calculations, the aggregate surface area for a dispersion of gas bubbles increases by a factor of 10 as the radius of the bubbles decreases by a factor of 10. Referring to calculations (b) and (c), and within rounding error, a dispersion of 50-micron diameter bubbles containing one cubic foot of gas will have an aggregate surface area more than 500 times greater than a dispersion of one-inch bubbles containing the same volume of gas; and a dispersion of 5 μm diameter bubbles containing one cubic foot of gas will have an aggregate surface area 10 times greater than the 50-micron diameter bubbles and 5,120 times greater than a dispersion of one-inch bubbles containing the same volume of gas.

Water having a desired temperature is pumped through an oxygenation system. More specifically, that water is conveyed through a pre-charge pump to pressurize it, preferably between 35 to 450 psig, more preferably around 120 to 150 psig. In addition, the water preferably has a temperature no greater than 65° F., as warmer temperatures decrease the solubility of the gas in solution and may not be appropriate for the medical condition being treated. The water discharged from that pre-charge pump is next conveyed to the point of gas introduction, or phase contactor, through an influent line maintained at low pressure. Oxygen-containing gas is introduced into the influent line from a supply of gas.

Once the dissolved gas has been “added”, the solution is fed into the topical bandage system shown and described through purposefully directional exit tube means. The dissolved oxygen in that solution will contact with the human wound (tissue) where it is believed it will assist with the regeneration of new tissue cells. It is further believed that such gaseous delivery will help the healing gases enter the bloodstream through capillaries. Such DIRECT wound contact the skin should exceed the equivalent benefit to a wound being exposed to pure oxygen in an oxygen tent.

Additional energy may be added to the solution to stimulate nucleation of micro-bubbles and accelerate a wound exfoliation process. Mechanical mixing or circulation of the solution with stirring bars, circulation pumps or other mechanical devices, prior to passage through the bandage and into or adjacent the wound may further stimulate the nucleation of micro-bubbles. In addition to the foregoing, still other stimulation enhancements may include adding one or more localized ultrasonic transducers.

Ordinarily, solution would be continuously introduced through the bandage and at or near the wound site before exiting through a bandage draining system or “sink”. Such one-pass systems are desirable for maximizing sanitation.

Referring now to FIG. 1B, there is shown a first alternative bandage device 110 over wound W on skin S. This first variation differs from that in FIG. lA in that it has multiple delivery portals 120a and 120b, the former having just one outlet 122a and the latter being further split into two distinct outlets 122b1 and 122b2. Also, in this first alternative embodiment, there is no single perimeter strip of adhesive. Rather, the entire underside to bandage 110 is coated with an adhesive material 118 and then gel G would be applied over the wound W for preventing unnecessary/undesirable adhesion directly to the wound itself. All other elements therein are consistent with FIG. 1A.

The solution discharged to wound W beneath the various bandage embodiments is conveyed through a directional tipped outlet, exit or end tube.

The piping for directional outlet may be made from any suitable material that permits the flow of a liquefied gas there through. Suitable materials are materials comprised of porous stainless steel, copper and alloys, nickel alloys, ceramics (Si₃N₄), porous carbon and titanium.

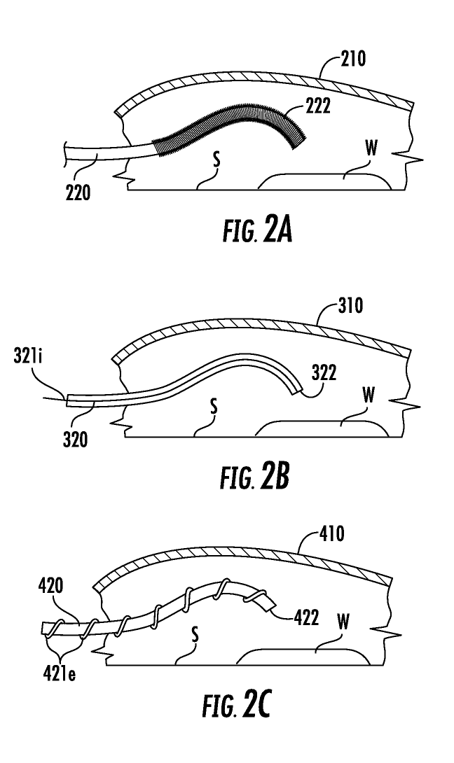

FIGS. 2A through 2C depict, in an enlarged cross-sectional view, how various outlets can be made flexibly adjusting to better deliver solution to or adjacent (or even partially into) a given wound site W on the skin S for treatment hereby. In FIG. 2A, delivery portal 220 has an accordion-shaped, plastic tube outlet end 222 that can be bent or manipulated (even after installation of the whole bandage 210 over the wound. Such directional adjustments will provide a better, preferred spray end (or wound contact) solution delivery point.

For alternative FIG. 2B, a more standard delivery portal tube 320 is used rather than the baffled, accordion-like arrangement of FIG. 2A. Thereafter, a stiffener component is wrapped completely there around. In FIG. 2B, that is an internal stiffening rod 321i (though in still other alternate variations, rod 321 may be replaced by or supplemented with a wire wrap or other segment that enables easy manipulation to the preferred wound contact site.

FIG. 2C differs from FIG. 2B in that its standard delivery tube 420 is provided with an external stiffener 421e for also accomplishing better directional solution delivery to the given wound site being treated.

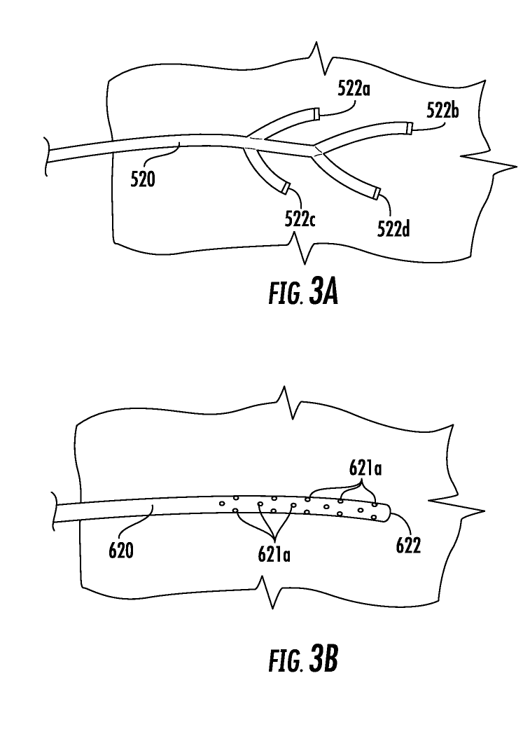

Still other outlet variations are depicted in accompanying FIGS. 3A and B. It is to be understood that the drain sink in each embodiment may be fitted with similar tube inputs. Particularly, at FIG. 3A, there is shown in top view a portal 520 having an intentionally split outlet end. As shown, that end can be divided into a plurality of outlet tips 522a, 522b, 522c and 522d. For the final alternative variation depicted, FIG. 3B includes a single portal 620 but one having a plurality of intermediate, multiple exit apertures 621a prior to reaching outlet tip 622.

In some instances, the tube proper may be formed from a metal, composite or plastic material. Preferably, the stiffener extending there through and/or thereabout is formed from stainless steel or Teflon®, titanium, nickel alloys, or some variation of flexible ceramic.

The terms and expressions, which have been employed herein, are used as terms of description and not of limitation. There is no intention in use of such terms and expressions of excluding any equivalents of the features shown and described or portions thereof. It is recognized that various modifications of the embodiments described herein are possible within the scope and spirit of the invention.

The invention is intended to encompass a wide range of solutes and solvents other than oxygen and water. For instance, injecting nitrogen gas into a solvent can form a two-phase mixture in accord with the present invention. A bath solution may be prepared using one or more gases, including, but not limited to air, carbon dioxide, nitrous oxide or a number of inert gases. Still other treatments may be realized by sequentially treating individuals in multiple gas phases. For instance, an initial treatment with CO₂, followed by O₂ and then, lastly N₂O.

Reference herein to oxygen is meant to include molecular oxygen, but reference to molecular oxygen is meant to include only molecular oxygen or diatomic oxygen, or non-free radical.

Having described the presently preferred embodiments, it is to be understood that the invention may be otherwise embodied within the scope of the appended claims.

What is claimed is:

1. A directional bandage for treating a human tissue wound by delivering a dissolved gas solution into and/or adjacent the wound, said directional bandage comprising:
a main body having an interior for covering the wound;
waterproof means for adhering the main body over the wound;
at least one delivery portal having an outlet through which a gas-containing solution may be delivered into and/or adjacent the wound, said portal having means for directing solution delivery to a particular area of the wound after the main body has been installed thereover; and
at least one drain sink for collecting and removing solution for disposal after it has been directionally delivered to the wound.

2. The directional bandage of claim 1 wherein the direction means includes an accordion-tipped tube end that can be bent in multiple directions.

3. The directional bandage of claim 1 wherein the direction means includes a malleable stiffener inside the delivery portal.

4. The directional bandage of claim 1 wherein the direction means includes a bendable stiffener about an exterior of the delivery portal.

5. The directional bandage of claim 1 wherein the delivery portal includes a plurality of apertures through which the solution is delivered to the wound.

6. A method of treating a human tissue wound to revitalize said wound, said method comprising:
(a) providing a bandage device that comprises: a main body having an interior for covering the wound; waterproof means for adhering the main body over the wound; at least one delivery portal having an outlet through which a gas-containing solution may be delivered into and/or adjacent the wound, said portal having means for directing solution delivery to a particular area of the wound after the main body has been installed thereover;
and at least one drain sink for collecting and removing solution for disposal after it has been directionally delivered to the wound;
(b) providing a skin surface with a wound requiring treatment;
(c) directing the delivery portal of the bandage device to a region at or near the wound;
(d) dissolving a gas in liquid to form a solution under elevated pressure for saturating or supersaturating dissolved gas into said solution; and
(e) revitalizing the wound with directional delivery of the gas there against.

7. The method of claim 6 wherein said solution contains nucleated gas micro-bubbles.

8. The method of claim 6 wherein said gas consists essentially of oxygen.

9. The method of claim 6 wherein said gas is selected from the group consisting of:
oxygen, air, nitrogen, carbon dioxide, nitrous oxide, nitric oxide, argon and combinations thereof.

10. The method of claim 6 wherein the solution further contains one or more compatible antibiotics.

11. The method of claim 6 wherein the solution further contains one or more compatible anesthetics.

12. A method of treating a human tissue wound to revitalize said wound, said method comprising:
(a) providing a bandage device that comprises: a main body having an interior for covering the wound; waterproof means for adhering the main body over the wound; a plurality of delivery portals, each having an outlet through which a gas-containing solution may be delivered into and/or adjacent the wound, said portals having means for directing solution delivery to a particular area of the wound after the main body has been installed thereover; and at least one drain sink for collecting and removing solution for disposal after it has been directionally delivered to the wound
(b) providing a skin surface with a wound requiring treatment;
(c) directing one or more delivery portals of the bandage device to preferred regions at or near the wound;
(d) dissolving a gas in liquid to form a solution under elevated pressure for saturating or supersaturating dissolved gas into said solution;
(e) revitalizing the wound with directional delivery of the gas there against;
(f) controlling the pressure within the space bound by the said bandage device and said skin surface.

13. The method of claim 12 wherein said pressure control is accomplished by manipulating incoming and/or outgoing flow.

14. The method of claim 12 wherein said pressure control is accomplished by using an outflow scavenge pump.

15. The method of claim 12, which further comprises:
controlling the temperature of said liquid during or after dissolving step (d).

16. The method of claim 15 wherein said temperature control is accomplished by a Peltier Effect device capable of heating and/or cooling

17. The method of claim 15 wherein said temperature control is accomplished by a Rankine Cycle compression-expansion device capable of heating and/or cooling.

18. The method of claim 12, which further comprises:
providing the exit tube on the bandage device with directional dispersion means for improving distribution of said liquid over the said skin surface or within a cavity.

19. The method of claim 18 wherein said directional dispersion means includes a porous medium.

20. The method of claim 18 wherein said directional dispersion means a tube to supply said liquid concentric with a porous medium to scavenge said liquid from the said skin surface.
